(19) United States
(12) Patent Application Publication
(10) Pub. No.: US 2019/0209290 A1
(43) Pub. Date: Jul. 11, 2019

(54) DEVICE AND PROCESS OF REMOVING VISCOUS AND FLUID MATERIALS FROM A HUMAN BODY CAVITY

(71) Applicant: YMB Medical, Inc., Milwaukee, WI (US)

(72) Inventors: Nazih John Yousif, Mequon, WI (US); Hani S. Matloub, Wauesha, WI (US); Michael Bamberger, Bayside, WI (US)

(21) Appl. No.: 16/243,147

(22) Filed: Jan. 9, 2019

Related U.S. Application Data
(60) Provisional application No. 62/615,121, filed on Jan. 9, 2018.

(51) Int. Cl. A61F 2/12; A61L 27/22; A61M 1/00

(52) U.S. Cl. CPC A61F 2/12; A61B 2017/00796; A61M 1/0001; A61L 27/222

(57) ABSTRACT

A method and device for removing the gelatinous filler of a ruptured breast implant is disclosed. The device has a large bore collection nozzle with large internal diameter and a short length so that it can transport a viscous gel using the suction pressure drop typically available in a hospital operating room and a collection container attached to the nozzle that has an internal volume to accommodate the filler. The container has a suction fitting for communication with a hospital suction port distal from the collection container and grasping means for manipulating the container.

### SUMMARY OF THE INVENTION

One embodiment of the present invention involves a device and process for removing the gelatinous filler of a ruptured breast implant. The device has a large bore collection nozzle with a sufficiently large internal diameter and a sufficiently short length to allow the transport of a gel having the viscosity of a typical silicone gel based breast implant filler using the suction pressure drop typically available in a hospital operating room. A collection container is attached to or is integral with the nozzle and has a sufficient internal volume to accommodate the total amount of filler typically found in a silicone breast implant wherein the passageway presented to the gelatinous filler increases substantially at the point at which the nozzle joins the collection device. This collection container has a suction fitting adapted to interact with a suction line and communicates in a suction tight manner with the interior of the collection device at a point distal from the point at which the nozzle joins the collection container. This collection container also has a grasping means by which the collection container and its attached nozzle may be readily manipulated.

Another embodiment of the invention involves a process of providing the collection device and using it to remove the gelatinous filler of a ruptured breast implant. The suction fitting is connected to a source of suction sufficient to transport the gelatinous filler from the mouth of the collection nozzle to the interior of the collection container. The device is manipulated by its grasping means so as to bring the collection nozzle immediately adjacent to the gelatinous filler of a ruptured breast implant and the nozzle is held in place in place until the bulk of the gelatinous filler has been transported to the interior of the collection container.

A further embodiment of the invention involves a process of providing a collection device and using it to collect a fluid material from an open body cavity of a human patient. In this regard, for the purposes of this disclosure the term body cavity is used to designate any portion of the human body in which blood, pus or another bodily fluid can pool such as an incision or recess accessed by a surgical incision. The collection device comprises a large bore collection nozzle with a sufficiently large internal diameter and a sufficiently short length to allow the transport of the fluid material using the suction pressure drop typically available in a hospital operating room substantially more rapidly than is achieved using a standard hospital suction system comprising a fluid collection canister which is located some distance from the patient, who is typically on an operating table, connected by lengthy tubing to a small bore collection nozzle. Optionally a collection nozzle extension is provided that can be affixed to the end of the collection nozzle distal from the collection container from which the collection nozzle extends. A collection container is attached to or is integral with the nozzle and has a sufficient internal volume to accommodate the total amount of the fluid to be collected. The passageway presented to the fluid material being collected presents a passageway that has no sharp turns and has a substantial increase in cross-section at the point at which the nozzle joins the collection device. A suction fitting adapted to interact with a suction line and communicate in a suction tight manner with the interior of the collection device is attached to the collection container at a point distal from the point at which the nozzle joins the collection container. The collection container is provided with grasping means by which the collection container and its attached nozzle may be readily manipulated. The suction fitting is connected to a source of suction sufficient to transport the fluid material from the mouth of the collection nozzle to the interior of the collection container. The device is then manipulated by its grasping means so as to bring the mouth of the collection nozzle or its extension immediately adjacent to the fluid material. Then nozzle or its extension is held in place until the bulk of the fluid has been transported to the interior of the collection container.

The ability of this embodiment to much more rapidly aspirate liquids or other bodily fluids from the open human cavity is of substantial clinical value. For instance, it can be of critical importance to rapidly assess the source of pooled blood in an open human cavity, as it may identify a condition, such as internal bleeding or hemorrhaging, that needs to be rapidly addressed. The significantly faster clearance may be attributed to both the larger bore of the collection nozzle and the short length of the collection nozzle. This could be viewed as an application of the principle of fluid mechanics that fluid throughput per unit time in a pipe increases with the ratio of the diameter of the pipe to its length. Of course, holding capacity or volume of the collection container is also of importance, as it is desirable not to overwhelm it with the aspirated liquid or other fluid. If there is a concern with unintentionally aspirating tissue or other solid materials, the collection nozzle or an extension of the collection nozzle may be provided with a filtering member that limits the size of the materials that can be aspirated. This filtering member can be any means that restricts access to the bore of the nozzle or its extension such as a cap with a fenestration pattern in its end wall or a cap with slits in its end wall and the portion of its sidewall that extends beyond the end of the nozzle or its extension.

### BRIEF DESCRIPTION OF THE DRAWINGS

FIG. 1 is a perspective view of the collection container with its collection nozzle and suction fitting.

FIG. 2 is a side elevation of the collection container with its collection nozzle and suction fitting.

FIG. 3 is a cross section of the collection container with its collection nozzle and suction fitting taken along section line 3-3 of FIG. 2 with volumetric markings shown.

FIG. 4 is a side elevation of the collection container with sealing caps affixed to both the collection nozzle and the suction fitting.

FIG. 5 is a side elevation of the collection container with a removable extension affixed to its collection nozzle.

FIG. 6 is a cross section of the collection container with a removable extension affixed to its collection nozzle taken along section line 6-6 of FIG. 5.

FIG. 7 is a prospective view of a collection container adapted to the collection of bodily fluids by the addition of a filtering member.

FIG. 8 a prospective view of a collection nozzle extension adapted to the collection of bodily fluids by the addition of a filtering member.

### DETAILED DESCRIPTION

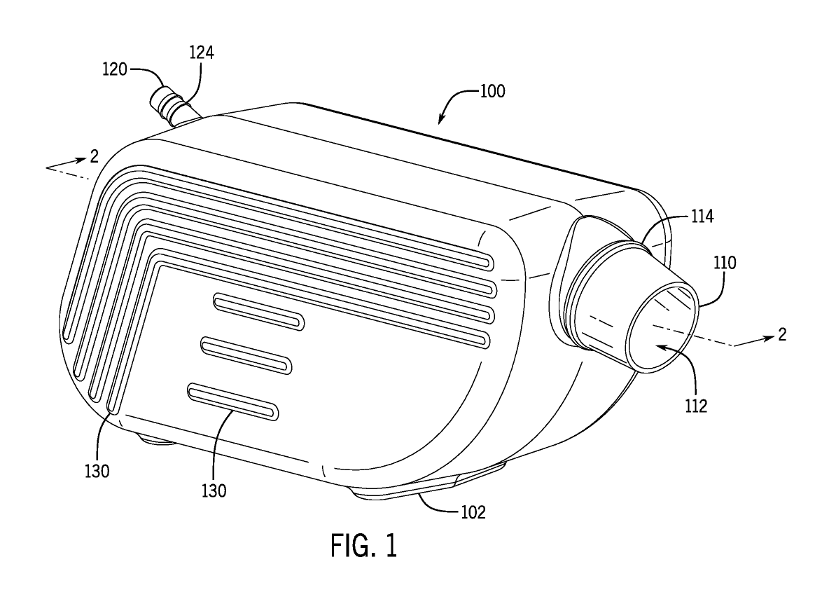

FIG. 1 illustrates a collection container 100 suitable to remove the gelatinous filler of a ruptured breast implant. The device is used by connecting a source of suction or vacuum to the suction fitting 120, securing the connection using the suction fitting boss 124. If the device is used in the typical hospital operating room (OR) the available suction will be about 21 inches of mercury, though suctions up to a full vacuum of around 29.9 inches of mercury may be conveniently available. The device is then manipulated by a surgeon using the grasping means 130, which are embossed ridges in the illustrated embodiment, to move the collection nozzle 110 into close proximity of the gelatinous filler. The suction will then cause this gelatinous filler to move into and through the bore 112 of the collection nozzle 110 and into the collection container 100. The collection container 100 conveniently has a volume large enough to accommodate the commonly encountered silicone breast implant fillers that range between about 125 and 800 ml, with volumes between about 200 and 500 ml being the most commonly encountered. Thus a volume of about 1,000 ml is convenient and provides a margin of safety though volumes up to about 1,750 ml or greater are useful. The volume should be constrained to avoid making the collection container 100 unnecessarily more difficult to manipulate. The collection container 100 may be provided with feet 102 to aid in allowing the collection container 100 to be sit stably on a flat surface such as a table before and after use. The collection nozzle 110 is provided with a boss 114 which serves several functions as will be discussed below. The collection nozzle 110 is illustrated as being integral with the collection container 100, but it could readily be a separate piece appropriately affixed to the collection container 100. This would facilitate making this nozzle 110 of a different material and/or giving it a thicker wall than that of the collection container 100.

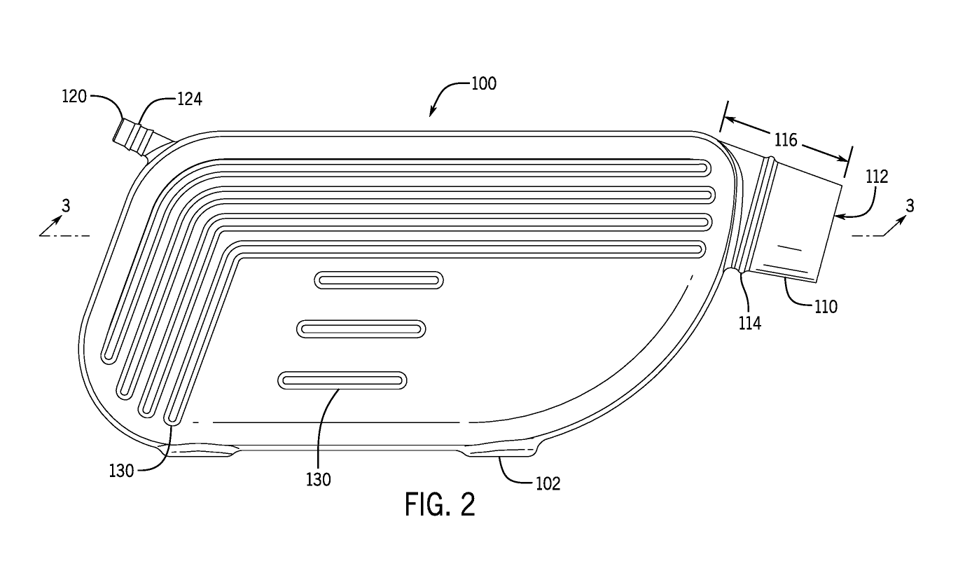

FIG. 2 is a further illustration of the collection container 100 with all of the features discussed in connection with FIG. 1. In addition it illustrates the collection nozzle length 116. It is convenient to minimize this length to facilitate the transport of a silicone gel breast implant filler. These fillers are now in their fifth generation with the later generations having significantly greater viscosities to minimize leakage. It would be particularly convenient if this length were short enough to facilitate the transport of the filler of the Natrelle Style 410 Implant from Allergan, which is generally acknowledged as the stiffest gel with the highest cohesivity of the fillers currently in use. However, there is substantial utility in devices that can transport the filler used in any of the implants from the non-form-stable Natrelle Truform 1, Mentor Cohesive Gel 1 and the Mentor Cohesive Gel 2 to the form stable Natrelle Truform 2, Mentor Cohesive Gel III and the Natrelle Truform 3. A length between about one and two inches is particularly convenient. The passageway presented to the fluid material being collected, such as the breast implant filler, has no sharp turns and has a substantial increase in cross-section at the point at which the collection nozzle 110 joins the collection device 100. In this regard, it is contemplated that an apparatus that readily transports these specifically identified gels will have substantial utility with other existing gels and future higher cohesivity gels that may be developed to serve as breast implant filler.

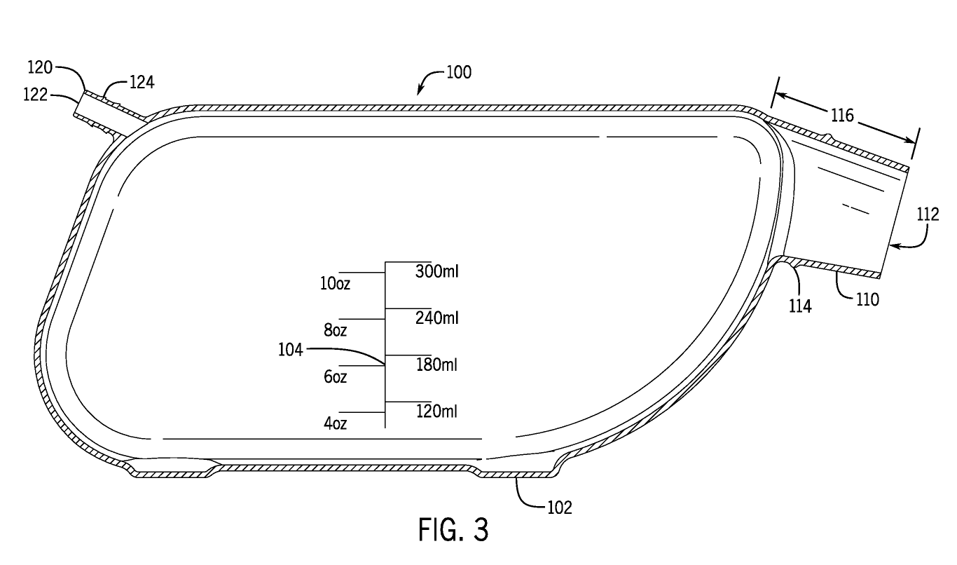

FIG. 3 is a further illustration of the collection container 100 with all of the features discussed in connection with FIG. 1 and FIG. 2. The inside bore 112 of the collection nozzle is seen in cross section as is the suction fitting bore 122. As with the length of the collection nozzle, its bore 112 is conveniently sized to facilitate the transport of a silicone gel breast implant filler. In this case the bore is conveniently not only large to facilitate transport but also small enough to allow the gelatinous filler from a single breast implant to form a vacuum seal with the inside wall of the collection nozzle 110. It has been observed that particularly efficient transport occurs when the filler essentially acts as a temporary plug in the collection nozzle 110. The collection nozzle bore 112 is conveniently between about one and two inches in diameter with diameters between about 1.2 and 1.4 inches being particularly efficacious. The bore 122 of the suction fitting 120 conveniently has a diameter less than one half of that of the collection nozzle bore 112. Its function is merely to provide suction and so need only transport the fluid, most typically air, providing the suction. Values is the range of about 0.25 inches are suitable to interact with typical vacuum lines, such as those found in hospital operating rooms. It is apparent that the collection nozzle 110 is located in a sidewall of the collection container opposite the side wall in which the suction fitting is located. This is a particularly convenient arrangement both from the point of view of minimizing the risk that any of the gelatinous filler could reach and potentially block the bore 122 of the suction fitting 120 and in terms of facilitating the manipulability of the collection container 100. A collection container 100 with a height between about 4 and 5 inches and a length between about 7 and 8 inches is convenient for allowing the collection nozzle 110 to be placed in the upper half of a sidewall of the collection container 100 and still have an advantageous bore 112 and allowing good separation between the collection nozzle 110 and the suction fitting 120. If these height and length dimensions are combined with a width between about 3 and 4 inches, a particularly manipulatable collection container 100 is obtained. It is advantageous if the dimensions of the collection container 100 are such that the collection nozzle 110 may be readily placed at a site of collection by a surgeon using a single hand. It is advantageous if one or more of the walls of the collection container 100 are transparent so that the collection of the silicone gel breast implant filler can be observed as it occurs. The collection container 100 can then be provided with volumetric markings 104 so that the volume collected at any point in time can be conveniently assessed.

The collection container 100 illustrated in FIG. 1-FIG. 3 is generally a rectangular prism, which makes it readily stackable for convenient storage, particularly in sterile packaging. The collection container 100 could have any convenient shape that provides the needed internal volume, adequate separation between the port that provides entry for the nozzle 110 and the port that provides entry for the suction fitting 120 and an increase in cross section at the port that provides entry for the nozzle 110. However, it is convenient if it has an overall shape that makes it readily stackable and packable in storage containers. This would provide for convenient storage in an environment with limited space, such as a hospital operating room.

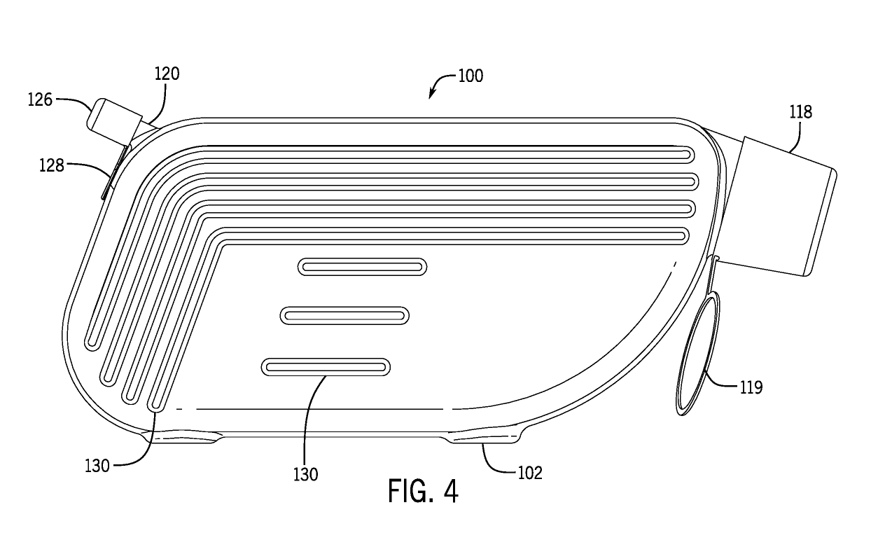

FIG. 4 is a further illustration of the collection container 100 with some of the features discussed in connection with FIG. 1, FIG. 2 and FIG. 3 and the added features of collection nozzle cap 118 with its retaining ring 119 and suction fitting cap 126 with its retaining ring 128. The caps 118 and 126 make it convenient to remove the collection container 100 from the site of use, such as a hospital operating room, while minimizing the risk that any of the collected contents will spill. The collection nozzle cap 118 may interact with the collection nozzle boss 114 (illustrated in FIG. 1-3) for a secure attachment to the collection nozzle 110 and the suction fitting cap 126 may similarly interact with the suction fitting boss 124 (also illustrated in FIG. 1-3) for a secure attachment to the suction fitting 120. The retaining rings 119 and 128 may interact with the bosses 114 and 124 to keep the caps 118 and 126 associated with the collection container 100 before the collection of the filler or other material is complete. The collection container 100 may conveniently be made of an inexpensive plastic material such as high density polyethylene (HDPE), low density polyethylene (LDPE), polyethylene terephthalate (PET), polyvinyl chloride (PVC) or polystyerene (PS) so that once it is filled, it can be provided with caps 118 and 126. The capped collection container can then be disposed of as medical waste or shipped to the implant manufacturer for investigation of the cause of the rupture. Alternatively it can be made of a more robust autoclavable material such as polypropylene (PP) and be emptied after use and then reused. In this case, the caps 118 and 126 just facilitate its transportation to a reconditioning site. It is convenient to form the collection container 100 from a readily formable plastic so that efficient fabrication techniques such as blow molding or injection molding may be employed in making it. The walls of the collection container 100 may be made fairly thin thus facilitating its fabrication, reducing its cost and minimizing its weight to enhance its manipulability. In cases in which these walls are made of a fairly flexible material and/or are relatively thin, the caps 118 and 126 may conveniently be made more rigid by material selection and/or greater wall thickness.

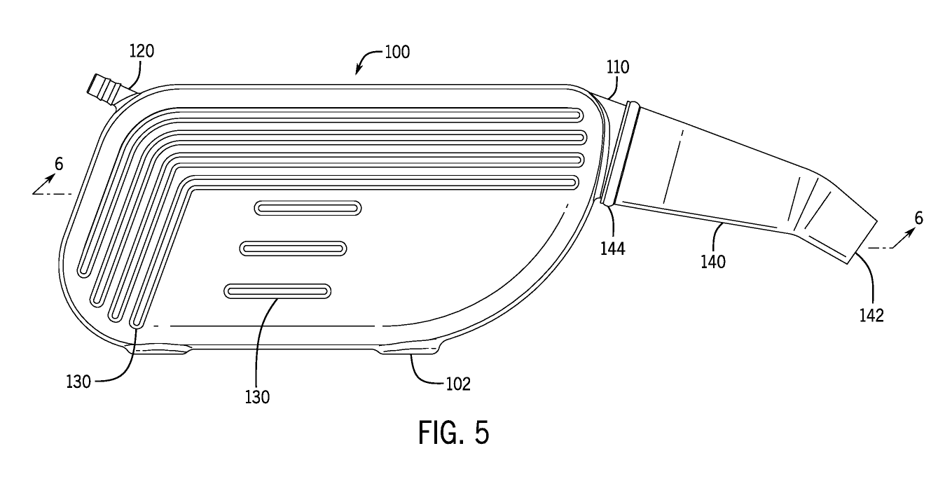

FIG. 5 is a further illustration of the collection container 100 with some of the features discussed in connection with FIG. 1, FIG. 2 and FIG. 3 and the added feature of a collection nozzle extension 140. This extension 140 has a bore 142 that is typically smaller than collection nozzle bore 112 and has a collar 144 which interacts with the collection nozzle boss 114 the secure the extension 140 to the nozzle 110 and the collection container 100. The extension 140 can be conveniently used to collect residual gelatinous filler from a ruptured breast implant or other materials of a fluid or semi-fluid nature that may be found in the interior of the human body. Because in this arrangement the distance from the collection point at the mouth of the extension 140 to the interior of the collection container 100 is minimized, the so configured device is advantageous for the collection of non-viscous materials such blood or purulent materials such as pus. The relevant fluid mechanics formulae indicate that the velocity and volumetric throughput rate of a pipe increases with increases in the ratio of the pipe diameter to its length. Thus short wide pipes such as the extension 140 should offer a significant advantage in collecting any material, viscous or non-viscous, using vacuum over the use of a long small bore vacuum line attached to a distant suction canister, the set up typical of an operating room suction collection system. In this regard, the extension typically extends beyond the end of the collection nozzle 110 by between about one and four inches and has a bore 142 of between about three quarters of an inch and three inches with an extension of about 2.6 inches and a bore of about 1.3 inches being particularly advantageous. The combination of the collection nozzle 110 and the collection nozzle extension 140 advantageously presents a passageway to the fluid material being collected that has no sharp turns and has a substantial increase in cross-section at the point at which the nozzle 110 joins the collection device 100. The efficient use of the extension 140 suggests that it have greater rigidity than the collection container 100 when the collection container 100 has been designed to enhance its formability and manipulability by, for instance having fairly thin walls. This rigidity can conveniently be provided by an appropriate construction material selection and/or a thicker wall.

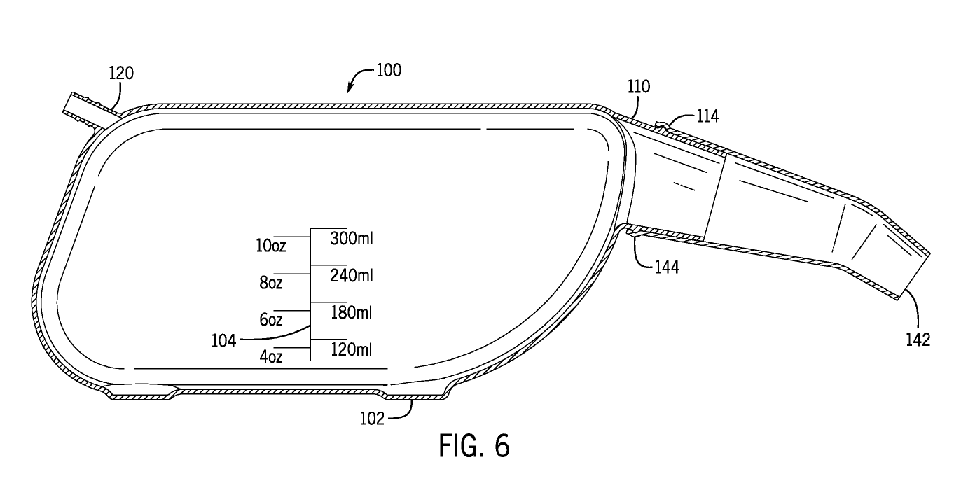

FIG. 6 is a further illustration of the collection container 100 with the features discussed in connection with FIG. 5 and the volumetric markings 104 discussed with regard to FIG. 3. As discussed with regard to FIG. 3, the volumetric markings 104 are most useful when one or more walls of the collection container 100 are transparent.

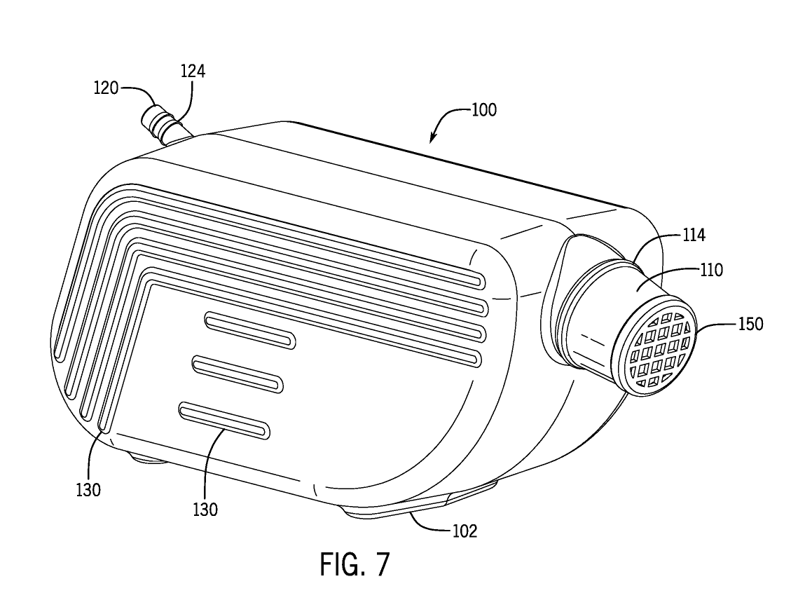

FIG. 7 is an illustration of a collection container 100 that has been adapted to more conveniently aspirate body fluids such as blood or pus by having its collection nozzle 110 fitted with a filtering member 150. The fenestration pattern shown is illustrative of an approach to preventing the aspiration of larger particles. However, any pattern that acts to separate fluid from solid or semi-solid would usefully aid in the aspiration of bodily fluids.

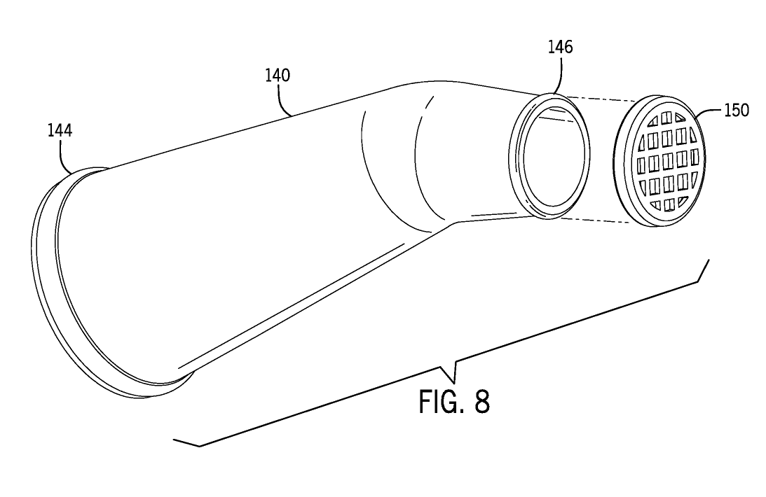

FIG. 8 is an illustration of a collection nozzle extension 140 that has been adapted to more conveniently aspirate body fluids such as blood or pus by being fitted with a filtering member 150. The fenestration pattern used in connection with the extension 140 can be the same of different from that used with the collection nozzle 110. The collection nozzle extension 140 has been provided with a boss 146 that interacts with the filtering member 150 to hold it securely in place. The collection nozzle 110 illustrated in FIG. 7 can conveniently be provided with a similar boss. The diameter of the filtering member 150 is conveniently selected to interact with either the collection nozzle 110 or the collection nozzle extension 140. Typically the end of the collection nozzle extension 140 distal from the collection container 100 is less than the diameter of the end of the collection nozzle 110 distal from the collection container 100. Thus a given filtering member 150 is adapted to one or the other.

While the filtering member 150 has been illustrated as a cap with an end wall with a fenestrated pattern, it could have a variety of designs for excluding larger particle from being collected. For instance, it could be a cap with side walls adapted to extend beyond the end of the nozzle 110 or nozzle extension 140 to which it is affixed with slits in both this portion of the side wall as well as in the end wall.

In this regard the, end wall could have a configuration other than planar such as semi-spherical or domed.

While only certain features of the invention have been illustrated and described herein, many modifications and changes will occur to those skilled in the art. It is, therefore, to be understood that the appended claims are intended to cover all such modifications and changes as fall within the true spirit of the invention.

What is claimed is:

1. A device for removing the gelatinous filler of a ruptured breast implant comprising:
a. a large bore collection nozzle with a sufficiently large internal diameter and a sufficiently short length to allow the transport of a gel having the viscosity of a typical silicone gel based breast implant filler using the suction pressure drop typically available in a hospital operating room;
b. a collection container attached to the nozzle which has a sufficient internal volume to accommodate the total amount of filler typically found in a silicone breast implant wherein the passageway presented to the gelatinous filler increases substantially at the point at which the nozzle joins the collection device;
c. a suction fitting adapted to interact with a suction line and communicate in a suction tight manner with the interior of the collection device at a point distal from the point at which the nozzle joins the collection container; and
d. a grasping means by which the collection container and its attached nozzle may be readily manipulated.

2. The device of claim 1 wherein the interior diameter of the nozzle is small enough to allow the gelatinous filler from a single breast implant to form a vacuum seal with the inside wall of the nozzle.

3. The device of claim 1 wherein the nozzle accesses the interior of the collection container via a port in a side wall of the collection container and the port is located in the upper half of this side wall.

4. The device of claim 3 wherein the suction fitting is located on the side wall of the collection container which is opposite the side wall containing the nozzle access port.

5. The device of claim 1 wherein the height of the container is at least about twice the interior diameter of the nozzle.

6. The device of claim 1 wherein the suction fitting accesses the interior of the collection container via a port whose diameter is less than one half of the interior diameter of the nozzle.

7. The device of claim 1 wherein at least one wall of the collection container is transparent such that the delivery of gelatinous filler into the collection container may be observed when the device is in use.

8. The device of claim 7 wherein the transparent side wall carries indicia by which the volume of filler removed by use of the device may be gauged.

9. The device of claim 1 wherein the device includes a removable extension that connects with the collection nozzle to extend its length and that has a reduced internal diameter at its end distal from the end adapted to connect to the collection nozzle.

10. A process for removing the gelatinous filler of a ruptured breast implant comprising:
a. providing a collection device comprising:
i. a large bore collection nozzle with a sufficiently large internal diameter and a sufficiently short length to allow the transport of a gel having the viscosity of a typical silicone gel based breast implant filler using the suction pressure drop typically available in a hospital operating room;
ii. a collection container attached to the nozzle which has a sufficient internal volume to accommodate the total amount of filler typically found in a silicone breast implant wherein the passageway presented to the gelatinous filler increases substantially at the point at which the nozzle joins the collection device;
iii. a suction fitting adapted to interact with a suction line and communicate in a suction tight manner with the interior of the collection device at a point distal from the point at which the nozzle joins the collection container; and
iv. a grasping means by which the collection container and its attached nozzle may be readily manipulated;
b. connecting the suction fitting to a source of suction sufficient to transport the gelatinous filler from the mouth of the collection nozzle to the interior of the collection container;
c. manipulating the device by its grasping means so as to bring the collection nozzle immediately adjacent to the gelatinous filler of a ruptured breast implant; and
d. holding the nozzle in place until the bulk of the gelatinous filler has been transported to the interior of the collection container.

11. The process of claim 10 wherein the process is performed in an operating room.

12. The process of claim 10 comprising the further steps of:
a. connecting an extension having one end with a reduced internal diameter to the collection nozzle such that this end is distal from the connection to the collection nozzle;
b. manipulating the collection device to bring this reduced internal diameter end adjacent to residual gelatinous filler not already transported to the interior of the collection container; and
c. holding the extension in place while this residual gelatinous filler is transported to the interior of the collection container.

13. The process of claim 10 wherein the interior diameter of the nozzle is small enough to allow the gelatinous filler from a single breast implant to form a vacuum seal with the inside wall of the nozzle.

14. The process of claim 10 wherein the nozzle accesses the interior of the collection container via a port in a side wall of the collection container and the port is located in the upper half of this side wall.

15. The process of claim 13 wherein the suction fitting is located on the side wall of the collection container which is opposite the side wall containing the nozzle access port.

16. The process of claim 10 wherein the height of the container is at least about twice the interior diameter of the nozzle.

17. The process of claim 10 wherein the suction fitting accesses the interior of the collection container via a port whose diameter is less than one half of the interior diameter of the nozzle.

18. The process of claim 10 wherein at least one wall of the collection container is transparent such that the delivery of gelatinous filler into the collection container may be observed when the device is in use.

19. The process of claim 17 wherein the transparent side wall carries indicia by which the volume of filler removed by use of the device may be gauged.

20. A process for collecting a fluid material from an open body cavity of a human patient comprising:
a. providing a collection device comprising:
i. a large bore collection nozzle with a sufficiently large internal diameter and a sufficiently short length to allow the transport of the fluid material using the suction pressure drop typically available in a hospital operating room substantially more rapidly than is achieved using a standard hospital suction system comprising a fluid collection canister which is located some distance from the patient connected by lengthy tubing to a small bore collection nozzle;
ii. optionally a collection nozzle extension that can be affixed to the end of the collection nozzle distal from the collection container from which the collection nozzle extends;
iii. a collection container attached to or integral with the nozzle which has a sufficient internal volume to accommodate the total amount of the fluid to be collected wherein the passageway presented to the fluid material has no sharp turns and has a substantial increase in cross-section at the point at which the nozzle joins the collection device;
iv. a suction fitting adapted to interact with a suction line and communicate in a suction tight manner with the interior of the collection device at a point distal from the point at which the nozzle joins the collection container; and
v. a grasping means by which the collection container and its attached nozzle may be readily manipulated;
b. connecting the suction fitting to a source of suction sufficient to transport the fluid material from the mouth of the collection nozzle to the interior of the collection container;
c. manipulating the device by its grasping means so as to bring the mouth of collection nozzle or its extension immediately adjacent to the fluid material; and
d. holding the nozzle or its extension in place until the bulk of the fluid has been transported to the interior of the collection container.

21. The process of claim 21 in which the nozzle or its extension is fitted with a filtering member to exclude larger size particles from being collected.
